(19) United States
(12) Patent Application Publication
(10) Pub. No.: US 2026/0182515 A1
(43) Pub. Date: Jul. 2, 2026

(54) Onion Variety 'BGS 390'

(71) Applicants: Bejo Zaden B.V., Warmenhuizen (NL); De Groot en Slot B.V., Broek op Langedijk (NL)

(72) Inventors: Timo Christiaan Petter, Warmenhuizen (NL); Jaap Jan Slot, Broek op Langedijk (NL)

(21) Appl. No.: 19/338,357

(22) Filed: Sep. 24, 2025

Related U.S. Application Data
(60) Provisional application No. 63/739,178, filed on Dec. 27, 2024.

(51) Int. Cl. A01H 6/04; A01H 5/04

(52) U.S. Cl. CPC A01H 6/04; A01H 5/04

(57) ABSTRACT

Provided herein is onion variety ‘BGS 390,’ including seeds thereof and methods of using the variety to generate additional onion varieties. The variety is characterized by its color, size, and shape.

### CROSS-REFERENCE TO RELATED APPLICATIONS

The present application claims priority to U.S. Provisional Patent Application No. 63/739,178, filed Dec. 27, 2024, the content of which is incorporated herein by reference in its entirety.

### BACKGROUND OF THE INVENTION

### Field of the Invention

Provided herein is a new variety of Allium fistulosum.

### Description of Related Art

Onion (Allium cepa) is an important crop, widely used in the cuisines of a variety of cultures. Moreover, onion is known to contain a number of important nutrients, including Vitamins B1, B2, and C, as well as minerals such as potassium and selenium. Furthermore, onion is known to have a number of beneficial effects, including in diabetes.

Bunching onion (Allium fistulosum) is a species of onion that very similar in taste and odor to the related A. cepa. However, A. fistulosum does not develop bulbs, and its leaves and scapes are hollow. A. fistulosum is mainly used as an ingredient in Asian cuisine, especially East Asia and Southeast Asia. It is particularly important in China, Japan, and Korea, and therefore is also called Japanese bunching onion.

### SUMMARY OF THE INVENTION

Provided herein is a seed of bunching onion variety ‘BGS 390,’ wherein a representative sample of seed of said variety was deposited under NCIMB Accession No. 44000.

Also provided herein are plants grown from seed of variety ‘BGS 390’, and plant parts and seeds produced by plants so grown.

Also provided herein are methods of generating offspring of variety ‘BGS 390’, including the steps of crossing a plant of variety ‘BGS 390’ with a second variety of bunching onion plant. In some embodiments, both plants are variety ‘BGS 390’. In other embodiments, the second variety is selected for a desired trait which can be introduced into offspring, such that said offspring include physiological/morphological traits of variety ‘BGS 390’ and the desired trait(s) of the second variety.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows onion variety ‘BGS 390’;

FIG. 2 shows onion variety ‘BGS 390’;

FIG. 3 shows onion variety ‘BGS 390’; and

FIG. 4 shows onion variety ‘BGS 390’.

### DESCRIPTION OF THE INVENTION

In the description which follows, a number of terms are used. To provide a clear and consistent understanding of the specification and claims, including the scope to be given for such terms, the following definitions are provided:

Amplified Fragment Length Polymorphism (AFLP®): A PCR-based method of identifying polymorphisms through digestion with known restriction enzymes, followed by visualization through use of radiography or fluorescence.

Allele: The allele is any of one or more alternative forms of a gene, all of which relate to one trait or characteristic. In a diploid cell or organism, the two alleles of a given gene occupy corresponding loci on a pair of homologous chromosomes.

Backcrossing: Backcrossing is a process in which a breeder repeatedly crosses hybrid progeny back to one of the parents, for example, a first generation hybrid F1 with one of the parental genotype of the F1 hybrid.

BC1: First backcross generation.

Cleaved Amplified Polymorphic Sequence (CAPS): A method of identifying genetic markers based on the length of restriction fragment lengths. Restriction fragments analyzed using CAPS are typically generated using a Restriction Fragment Length Polymorphism assay.

Cotyledon: One of the first leaves of the embryo of a seed plant, typically one or more in monocotyledons, two in dicotyledons, and two or more in gymnosperms.

Clustered Regularly Interspaced Short Palindromic Repeats (CRISPR): Refers to DNA sequences found in bacteria that include portions of DNA introduced to the bacteria by a given virus. The concept is the basis of the CRISPR system of editing an organism's genome (see, e.g., U.S. Pat. No. 8,697,359, incorporated herein by reference in its entirety).

CRISPR-Associated System (CAS): A set of homologous genes, encoding enzymes, that reside in the genome near the site of CRISPR sequences. These enzymes target DNA sequences based on similarity to the viral sequences included in the CRISPR regions.

Embryo: A plant embryo is a portion of the seed including precursors of the leaves, stem, and root, and one or more cotyledons.

F1, F2, F3 etc.: First, second, third, etc. filial generation of offspring of distinctly different parental types.

First water date: The date the seed first receives adequate moisture to germinate. This can and often does equal the planting date.

Gene: As used herein, “gene” refers to a segment of nucleic acid. A gene can be introduced into a genome of a species, whether from a different species or from the same species, using transformation or various breeding methods.

Genetically Modified Organism (GMO): An organism whose genome has been altered using some form of genetic engineering.

Hypocotyl: The portion of the stem of an embryo plant beneath the cotyledons, but above the root.

Marker-assisted recurrent backcrossing (MARB): A method of introducing a single locus of interest. The MARB method allows for maintenance of essential characteristics of the recurrent parent's genome. MARB is particularly effective for Quantitative Trait Loci (QTLs) that are highly variable.

Marker-assisted selection (MAS): A method of selecting a trait of interest based not on the trait, but on a marker associated with that trait.

Maturity date: Maturity refers to the stage when the plants are of full size or of optimum weight, in marketable form or in shape to be of commercial or economic value.

Meristematic cell: Cells of the meristem, which is a collection of undifferentiated cells in the plant. Meristematic cells include apical meristematic cells and lateral meristematic cells.

Peronospora destructor: A plant pathogen that causes downy mildew on leaves of cultivated and wild Allium.

Pest: A bacterium, fungus, virus, insect, or animal that attacks or negatively affects a plant.

Plant: “Plant” includes plant cells, plant protoplasts, plant cells of tissue culture from which bunching onion plants can be regenerated, plant calli, plant clumps, and plant cells that are intact in plants, or parts of plants such as pollen, flowers, seeds, leaves, stems, and the like.

Promoter: A region of DNA upstream from the start of transcription and involved in recognition and binding of RNA polymerase and other proteins to initiate transcription. A “plant promoter” is a promoter capable of initiating transcription in plant cells.

Protoplast: A plant cell that has had its cell wall at least partially removed.

Quantitative Trait Loci (QTL): Refers to genetic loci that control, to some degree, numerically representable traits that are usually continuously distributed.

Random Amplified Microsatellite Polymorphism (RAMP): A PCR-based method of identifying polymorphisms through use of simple sequence repeat markers and random amplified DNA polymorphism markers. RAMP is particularly useful for assessing genetic relationships in plant species.

Random Amplified DNA Polymorphism: A PCR-based method of amplifying random sections of DNA.

Ratio of head height/diameter: Head height divided by the head diameter is an indication of the head shape wherein a value of <1 is flattened, 1=is round, and >1 is pointed.

Recurrent Parent: The backcross parent, and member of an identifiable lineage or line that is improved by addition of a trait not found in that line.

Regeneration: Regeneration refers to the development of a plant from tissue culture.

Resistance: The character of a plant to restrict or even inhibit the development of a pest or pathogen in or on the plant and also the restriction of damage these organisms may cause in comparison to a susceptible variety and under comparable circumstances.

Restriction Fragment Length Polymorphism (RFLP): A marker in homologous DNA, detectable based on fragments of different, specific lengths generated by known restriction enzymes.

Root tip: The terminal portion of the root of a plant.

Royal Horticultural Society of England (RHS): An organization that publishes an official botanical color chart quantitatively identifying colors according to a defined numbering system. The chart may be purchased from Royal Horticulture Society Enterprise Ltd., RHS Garden; Wisley, Woking; Surrey GU236QB, UK. As used herein, references to RHS color refer to those in the Sixth Edition (2019) of the chart.

Simple sequence repeats (SSR): A DNA sequence having a number of motifs that are repeated.

Single nucleotide polymorphism (SNP): A common nucleotide variation at a given locus among a given population.

Transgenic: An organism that contains genetic material from an unrelated organism that has been artificially introduced.

### Bunching Onion Variety ‘BGS 390’

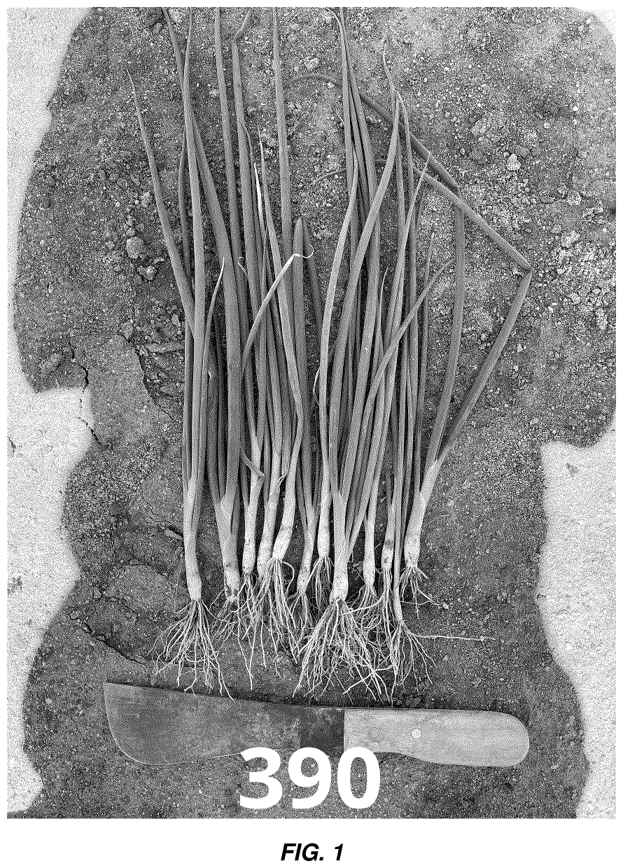

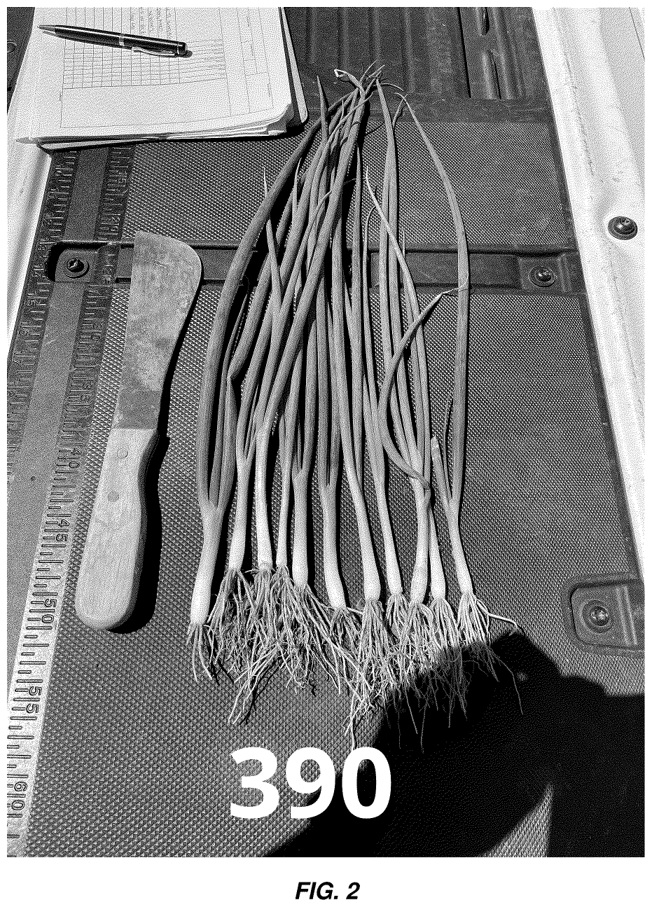

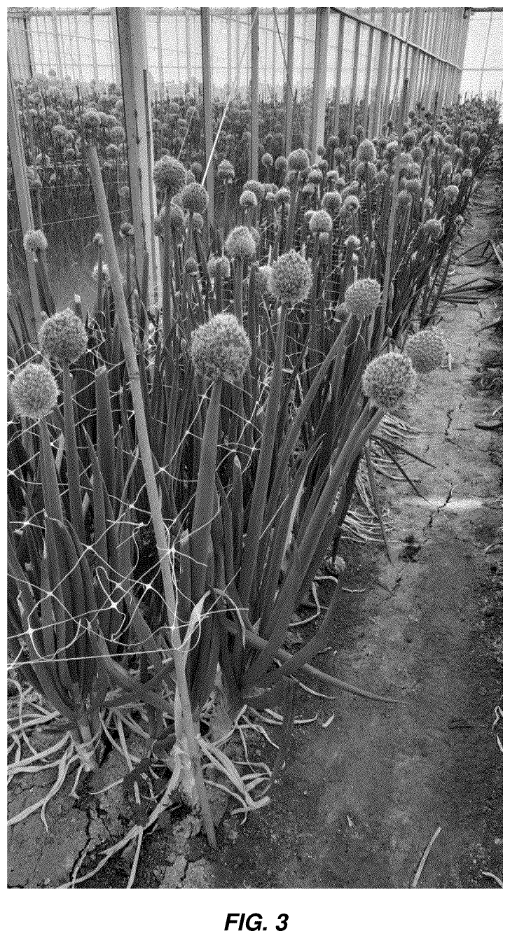

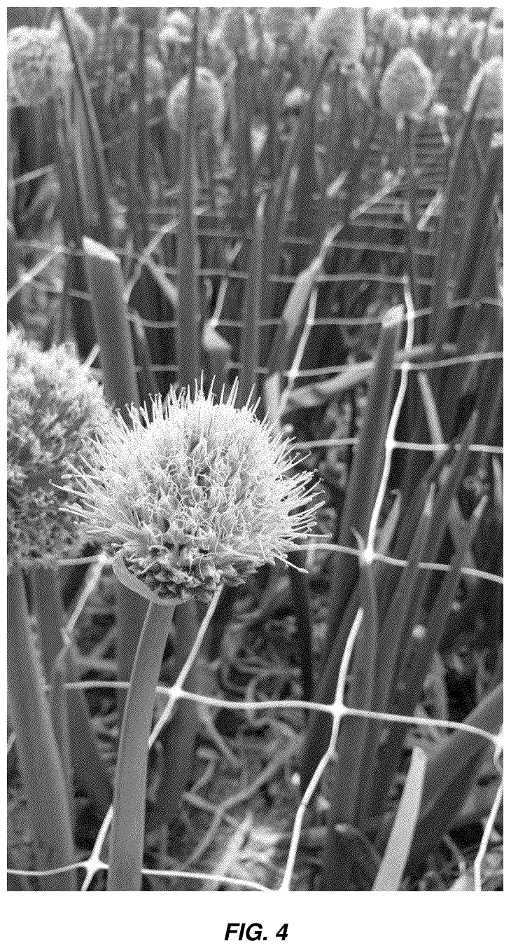

The following detailed description sets forth the characteristics of the new variety. Plants of the new variety were greenhouse grown in Broek-op-Langedijk, the Netherlands under normal (e.g., no additional) light, with temperatures ranging from approximately 18° C. to 40° C. (25° C. to 40° C. during the day and 18° C. to 25° C. at night). The color readings and measurements were taken in Boskoop, the Netherlands, under natural light on close to 3-month-old plants (˜80 days). Color references are primarily to the 2015 R.H.S. Colour Chart of The Royal Horticultural Society of London, Sixth Edition. Various aspects of the variety ‘BGS 390’ are shown in FIGS. 1-4 and are presented in Table 1 below.

Bunching onion variety ‘BGS 390’ is an herbaceous perennial and is a seed propagated bunching onion. Time from sowing to harvestable plant is approximately 12-13 weeks.

Bunching onion variety ‘BGS 390’ is similar to the variety ‘Baja Verde’, in that both are seed propagated varieties and show good heat tolerance. As used herein, “similar” or “similarity” is defined as the number of distinguishing characteristics that are the same between the two plants that are compared when grown under the same environmental conditions. Characteristics are considered “the same” when the value for a numeric characteristic is evaluated at significance levels of 1%, 5% or 10% significance level, or when a non-numeric characteristic is identical if the plants are grown under the same conditions. ‘Baja Verde’ tends to bulb more, while ‘BGS 390’ does not. The growth habit of ‘Baja Verde’ can express twisting, while growth habit of ‘BGS 390’ does so much less. Also, the foliage of ‘Baja Verde’ while upright has a more open habit, while the ‘BGS 390’ is noticeably more erect. When compared to other bunching onion varieties known to the Applicant, ‘BGS 390’ can be distinguished at least based its vigorous erect top displaying less twisting and bulbing in the pseudostem.

### Breeding History

The breeding history of ‘BGS 390’ is shown in Table 2. Briefly, individuals that have shown much less degree of bulbing and twisting and more vigor were selected from a population in Bakersfield, and were selected continuously until a uniform variety with desired characteristics was established.

A deposit of seeds of ‘BGS 390’ as disclosed above and recited in the appended claims has been made with the National Collections of Industrial, Food and Marine Bacteria (NCIMB), located in Wellheads Place, Dyce, Aberdeen, AB21 7 GB, United Kingdom. The date of the deposit was Jul. 22, 2022. All restrictions will be irrevocably removed upon granting of a patent, and the deposit is intended to meet all of the requirements of 37 C.F.R. § 1.801-1.809. The NCIMB Accession Number is 44000. The deposit will be maintained in the depository for a period of thirty years, or five years after the last request, or for the enforceable life of the patent, whichever is longer, and will be replaced as necessary during that period.

### Botanical Description

Characteristics of ‘BGS 390’ are provided in Table 1, below:

TABLE 1

| | |
|---|---|
| Overall plant shape: | Narrow obovate, with inflorescences growing from the |
| | base of the plant |
| Growth habit: | Upright |
| Plant height: | |
| Soil level to top of foliar plane: | Average: 84 cm |
| Growth rate (low, moderate or high): | Moderate |
| Plant vigor (low, moderate or high): | high |
| Cut flowers; number of harvested | N/A |
| stems/year: | |
| Branching habit: | Main flowering stems grow from the base, no lateral |
| | branches |
| Basal branching? | No branches present |
| Pinching required? | N/A |
| Number of primary (main) branches | No branches present |
| per plant: | |
| Number of secondary (lateral) | No branches present |
| branches per plant: | |
| Main stems: | |
| Length: | N/A |
| Diameter: | N/A |
| Stems: | |
| Appearance and shape: | N/A |
| Luster: | N/A |
| Aspect/angle from horizontal: | N/A |
| Strength: | N/A |
| Color, developing: | N/A |
| Color, mature: | N/A |
| Color at internodes: | N/A |
| Color of bark/older stems (if | N/A |
| present): | |
| Pubescence? | N/A |
| Other plant and/or stem | N/A |
| characteristics? | |
| Foliage: | |
| Leaf arrangement: | In basal rosettes, equitant |
| Compound or single: | Single |
| If compound, number of leaflets per | N/A |
| leaf: | |
| Number of leaves per lateral branch: | Average number of leaves per basal rosette: 4 |
| Whole leaf shape: | Terete, ensiform in outline |
| Whole leaf aspect: | Leaf blade terete (circular), hollow |
| Whole leaf length: | Average: 83 cm |
| Whole leaf width: | Leaf terete (circular), average diameter: 1.2 cm |
| Leaflet shape: | N/A |
| Leaflet length: | N/A |
| Leaflet width: | N/A |
| Leaf/leaflet apex: | Narrow acuminate |
| Leaf/leaflet base: | Sheathing |
| If sheathing, sheath length: | Average: 31.5 cm |
| If sheathing, sheath width: | Average: 4.7 cm |
| If sheathing, sheath color: | Transparent yellow-green; 145D and lighter, axially |
| | veined darker; 145A and 145B |
| Leaf/leaflet margin: | No margins present, leaves terete (circular) |
| Leaf/leaflet margin undulation: | None |
| Leaf/leaflet lobed? | Unlobed |
| If lobed, depth of sinuses (deep, | N/A |
| medium or shallow): | |
| Sinus orientation (divergent, parallel | N/A |
| or convergent): | |
| Leaf/leaflet texture, upper side: | Leaves terete (circular), outside smooth, glabrous |
| Leaf/leaflet texture, under side: | No leaf under sides present; leaves terete (circular) |
| Leaf/leaflet pubescence? Which | No leaf pubescence present, but leaf covered with a |
| side? | thin waxy layer, colored greyed-green; 189A |
| Leaf/leaflet rugosity? Which side? | Outside non-rugose |
| Leaf/leaflet luster, upper side: | Outside slightly glossy, but covered with a matte waxy |
| | layer |
| Leaf/leaflet luster, under side: | N/A; no leaf under sides present; leaves terete |
| | (circular) |
| Leaf/leaflet color, young, upper side: | Yellow-green; 144A |
| Leaf/leaflet color, young, under side: | N/A; no leaf under sides present; leaves terete |
| | (circular) |
| Leaf/leaflet color, mature, upper | Green; NN137B and 143A |
| side: | |
| Leaf/leaflet color, mature, under | N/A; no leaf under sides present; leaves terete |
| side: | (circular) |
| Venation pattern: | Parallel |
| Mature leaf venation color, upper | Green; NN137B and 143A |
| side: | |
| Mature leaf venation color, under | N/A; no leaf under sides present; leaves terete |
| side: | (circular) |
| If applicable, leaf/leaflet autumn | N/A |
| color, upper side: | |
| If applicable, leaf/leaflet autumn | N/A |
| color, under side: | |
| Petioles? | No leaves sessile (sheathing) |
| Petiole length: | N/A |
| Petiole diameter: | N/A |
| Petiole texture: | N/A |
| Petiole luster: | N/A |
| Petiole strength (high, medium, | N/A |
| low?): | |
| Petiole color, upper side: | N/A |
| Petiole color, under side: | N/A |
| Stipules, tendrils, thorns, spines, | None present |
| prickles? | |
| If present, please describe type, size, | |
| texture and color: | |
| Other leaf characteristics: | Yes, when crushed, the leaves spread an onion-like |
| | fragrance, typical for Allium |
| Inflorescence: | |
| Arrangement | Inflorescence |
| Inflorescence type and form: | Umbel, globular |
| Inflorescence height: | Average: 8.5 cm (excl. peduncle) |
| Inflorescence diameter: | Average: 8.6 cm |
| Quantity of flowers per | Average: 300 |
| inflorescence: | |
| Quantity of flowers per plant: | Average: approx. 150 |
| Quantity of flower buds per plant: | Average: approx. 150 |
| Quantity of flowers and flower buds | Average: approx. 150 |
| per plant: | |
| Natural flowering season: | Late spring into summer |
| Time to flower or response time: | Average time to flower after propagation: 6 months |
| Flower bud: | |
| Flower bud length: | Average: 6.0 mm |
| Flower bud diameter: | Average: 3.0 mm |
| Flower bud shape: | Ovate, triangular in cross-section |
| Flower bud texture: | Smooth, glabrous |
| Flower bud luster: | Slightly glossy |
| Flower bud color: | White; 155C, main veins of immature tepals green; |
| | 143C |
| Flower: | |
| Flower type and form: | Rotate, single |
| Flower aspect (upright, outwardly, | All directions |
| nodding, drooping): | |
| Flowering habit (freely, moderate or | Freely |
| low): | |
| Fragrance (strength and description): | Very faint, sweet and pleasant |
| Flower shape: | Rotate, consisting of two whorls of tepals |
| Flower diameter (horizontal): | Average: 1.0 cm |
| Flower diameter (vertical): | Average: 1.0 cm |
| Flower height (depth): | Average: 1.2 cm |
| Flower throat diameter (if | N/A |
| applicable): | |
| Flower throat texture (if applicable): | N/A |
| Flower tube length (if applicable): | N/A |
| Flower tube diameter (if applicable): | N/A |
| Flower tube texture (if applicable): | N/A |
| Flower longevity on plant: | Average: 3.5 weeks |
| Flower longevity as a cut flower (if | N/A |
| applicable): | |
| Persistent of self-cleaning? | Persistent |
| Petals: | Perianth consisting of tepals only, no separate petals |
| | and sepals but two whorls of tepals |
| Tepal arrangement: | Rotate |
| Tepal number (per flower): | Average: 6 |
| Number of tepal whorls: | Average: 2 |
| Tepals fused? | No |
| Tepal length: | Lower tepals average: 7.0 mm |
| | Upper tepals average: 8.0 mm |
| Tepal width: | Lower tepals and upper tepals average: 3.0 mm |
| Tepal shape: | Lower tepals ovate |
| | Upper tepals obovate to near oblong, slightly concave |
| Tepal tip: | Lower tepals and upper tepals acute |
| Tepal base: | Broad cuneate |
| Tepal margin: | Entire |
| Tepal margin undulation (none, low, | None |
| medium or high)? | |
| Tepal texture, upper side: | Smooth, glabrous |
| Tepal texture, under side: | Smooth, glabrous |
| Tepal rugosity, which side(s): | Both sides non-rugose |
| Tepal luster, upper side: | Slightly glossy |
| Tepal luster, under side: | Slightly glossy |
| Tepal color, when opening, upper | Lower tepals and upper tepals white; NN155D |
| side: | |
| Tepal color, when opening, under | Lower tepals and upper tepals white; NN155D |
| side: | |
| Tepal color, fully opened, upper | Lower tepals and upper tepals white; NN155D |
| side: | |
| Tepal color, fully opened, under | Lower tepals and upper tepals white; NN155D |
| side: | |
| Tepal venation, fully opened, upper | On lower tepals and upper tepals main veins green; |
| side: | 143C |
| Tepal venation, fully opened, under | On lower tepals and upper tepals main veins green; |
| side: | 143B to 143C |
| Tepal color, when fading, upper | Lower tepals and upper tepals greyed-white to green- |
| side: | white; a blend of 156D and 157D |
| Tepal color, when fading, under | Lower tepals and upper tepals greyed-white to green- |
| side: | white; a blend of 156D and 157D |
| Flower throat color: | N/A |
| Flower throat venation color: | N/A |
| Flower tube color: | N/A |
| Flower tube venation color: | N/A |
| Tepaloids? | No tepaloids present |
| Tepaloid arrangement: | N/A |
| Tepaloid number per flower: | N/A |
| Number of tepaloid whorls: | N/A |
| Tepaloids fused? | N/A |
| Tepaloid shape: | N/A |
| Tepaloid length: | N/A |
| Tepaloid width: | N/A |
| Tepaloid shape: | N/A |
| Tepaloid tip: | N/A |
| Tepaloid base: | N/A |
| Tepaloid margin: | N/A |
| Tepaloid margin undulation (none, | N/A |
| low, medium or high)? | |
| Tepaloid texture, upper side: | N/A |
| Tepaloid texture, under side: | N/A |
| Tepaloid rugosity, which side(s): | N/A |
| Tepaloid luster, upper side: | N/A |
| Tepaloid luster, under side: | N/A |
| Tepaloid color, when opening, upper | N/A |
| side: | |
| Tepaloid color, when opening, under | N/A |
| side: | |
| Tepaloid color, fully opened, upper | N/A |
| side: | |
| Tepaloid color, fully opened, under | N/A |
| side: | |
| Tepaloid venation, fully opened, | N/A |
| upper side: | |
| Tepaloid venation, fully opened, | N/A |
| under side: | |
| Tepaloid color, when fading, upper | N/A |
| side: | |
| Tepaloid color, when fading, under | N/A |
| side: | |
| Calyx: | |
| Calyx shape: | Rotate |
| Calyx length: | Average: 6.0 mm |
| Calyx diameter: | Average: 1.0 cm |
| Sepals: | No separate petals and sepals, only two whorls of |
| | tepals, see above |
| Sepal arrangement: | N/A |
| Sepal number per flower: | N/A |
| Number of sepal whorls: | N/A |
| Sepal length: | N/A |
| Sepal width: | N/A |
| Sepals fused? | N/A |
| Sepal shape: | N/A |
| Sepal tip: | N/A |
| Sepal base: | N/A |
| Sepal margin: | N/A |
| Sepal texture, upper side: | N/A |
| Sepal texture, under side: | N/A |
| Sepal luster, upper side: | N/A |
| Sepal luster, under side: | N/A |
| Sepal color, when opening, upper | N/A |
| side: | |
| Sepal color, when opening, under | N/A |
| side: | |
| Sepal color, fully opened, upper | N/A |
| side: | |
| Sepal color, fully opened, under | N/A |
| side: | |
| Tepal spurs? | No tepal spurs present |
| Number of tepal spurs per flower: | N/A |
| Tepal spur length: | N/A |
| Tepal spur diameter: | N/A |
| Tepal spur shape: | N/A |
| Tepal spur apex: | N/A |
| Tepal spur base: | N/A |
| Tepal spur texture: | N/A |
| Tepal spur luster: | N/A |
| Tepal spur color: | N/A |
| Peduncles? | Yes |
| Terminal peduncle length: | Average: 122.7 cm |
| Terminal peduncle diameter: | Peduncle flattened, average diameter at widest: 1.9 cm, |
| | average diameter at narrowest: 1.4 cm |
| Peduncle angle to soil level (= 0°): | Average angle: 80° |
| Peduncle strength: | Medium strong |
| Peduncle texture: | Smooth, glabrous, covered with a very thin waxy layer, |
| | greyed-green; 189A to 189B |
| Peduncle luster: | Moderately glossy, but not visible due to the very |
| | slightly glossy waxy layer covering the peduncle |
| Peduncle color: | Green; NN137A and NN137B |
| Pedicels? | Yes |
| Pedicel length: | Average: 2.8 cm |
| Pedicel diameter: | Average: 1.0 mm |
| Pedicel angle to peduncle axis: | Angle varying between: 0° to 160° |
| Pedicel strength: | Strong |
| Pedicel texture: | Smooth, glabrous |
| Pedicel luster: | Moderately glossy |
| Pedicel color: | Yellow-green; 144A to 144B |
| Floral bracts? If so, please provide | Yes, inflorescence bud covered by a papery bract, |
| type, size, texture and color: | splitting in two parts when inflorescence bud opens, |
| | average length: 4.2 cm, average width of each part: 1.7 |
| | cm, colored transparent greyed-white to orange-white; |
| | a blend of 156D and 159D, veined orange-white; 159B |
| Reproductive organs: | |
| Number of stamen per flower: | Average: 6 |
| Filament length: | Average: 7.0 mm |
| Filament color: | White; NN155C |
| Anther shape: | Basifixed, oblong |
| Anther length: | Average: 2.0 mm |
| Anther width: | Average: 1.0 mm |
| Anther color: | Yellow-green; 151D |
| Amount of pollen (none, low, | Low |
| moderate or abundant): | |
| Pollen color: | Yellow-green; 154D |
| Pistil number (per flower): | Average: 1 |
| Pistil length: | Average: 7.5 mm |
| Stigma shape: | Pointed |
| Stigma length: | Average: 0.5 mm |
| Stigma diameter: | Average: 0.2 mm |
| Stigma color: | White; NN155D |
| Style length: | Average: 7.0 mm |
| Style color: | White; NN155D |
| Ovary color: | Green; 138D |
| Fruit/seeds present? | No fruit/seed observed/present at time of harvest |
| Quantity of fruit per plant: | N/A |
| Fruit length: | N/A |
| Fruit diameter: | N/A |
| Fruit texture: | N/A |
| Fruit color: | N/A |
| Fruit taste: | N/A |
| Number of days to fruit ripening: | N/A |
| Fruit keeping quality (days) | N/A |
| Other fruit characteristics? | N/A |
| Quantity of seeds per fruit: | N/A |
| Seed length: | N/A |
| Seed diameter: | N/A |
| Seed texture: | N/A |
| Seed color: | N/A |
| Roots: | |
| Root structures (bulbs, corms, | No |
| rhizomes)? | |
| Shape of root structure: | N/A |
| Length of roots/root structure: | N/A |
| Diameter of roots/root structure: | N/A |
| Texture of root/root structure: | N/A |
| Color of root/root structure: | N/A |
| Miscellaneous: | |
| High temperature tolerance (° C.) | Unknown, but at least tolerant to temperatures up to 40 |
| | ° C. |
| Low temperature tolerance (° C.) | Unknown, at least hardy to USDA zone 5 (−23° C.) |
| USDA hardiness zones? | USDA zone 5 to USDA zone 10 |
| Tolerance to rain (none, low, | Low to moderate |
| moderate or high)? | |
| Tolerance to wind (none, low, | Low |
| moderate or high)? | |
| Pest (insect or arachnid) resistance | Similar to other perennial Allium varieties |
| or tolerance: | |
| (please provide genus and species) | |
| Disease resistance or tolerance: | Similar to other perennial Allium varieties |
| (please provide genus and species) | |
| Other plant or flower characteristics | No |
| not described above? | |

TABLE 2
Breeding history of BGS 390

| Country | | Male ♂ |
|---|---|---|
| USA | | Selected 50 individuals for inbreeding from |
| | | commercially available population |
| USA | | |
| USA | | Selected 10 best performing inbred lines in |
| | | field screening |
| USA | | Selected best performing inbred line nr 3 |
| USA | | Increasing ♂ inbred line nr 3 |
| USA | | Increasing ♂ inbred line nr 3 |
| USA/world | Testing line variety | Maintaining stable ♂ line |
| USA/world | Testing line variety | Maintaining stable ♂ line |
| USA/world | Testing line variety | Maintaining stable ♂ line |
| USA/world | Testing line variety | Bulking stable ♂ line |
| USA/world | Decision making BGS | Bulking stable ♂ line |
| | 390 | |

### FURTHER EMBODIMENTS—ADDITIONAL BREEDING METHODS

Also provided herein are methods for producing a bunching onion plant by crossing a first parent bunching onion plant with a second parent bunching onion plant, wherein the first or second parent bunching onion plant is a bunching onion variety disclosed herein. Plant breeding techniques, including crossing, thus crossing the bunching onion varieties disclosed herein with another plant (including the varieties disclosed herein), or with another variety (related or unrelated to the varieties disclosed herein), are well within the skill of the ordinary artisan. Thus, any such methods, e.g., selfing, backcrossing, hybrid production, crosses to populations, and the like, using the bunching onion varieties disclosed herein should be considered part of this invention. All plants produced using one or more of the bunching onion varieties disclosed herein as are within the scope of the disclosure, including those developed from varieties derived from the bunching onion varieties disclosed herein.

In aspects of a breeding method described herein, both first and second parent bunching onion plants are one or more of the bunching onion varieties disclosed herein.

In other aspects, the bunching onion varieties disclosed herein can be used in crosses with other, different bunching onion plants to produce the first generation (F1) bunching onion hybrid seeds and plants with superior characteristics. The bunching onion varieties disclosed herein can also be used for transformation where exogenous genes are introduced and expressed. Genetic variants created either through traditional breeding methods using the bunching onion varieties disclosed herein or through transformation of the bunching onion varieties disclosed herein by any of a number of protocols known to those of skill in the art are intended to be within the scope of the present invention.

In one aspect, the method includes the steps of: obtaining the bunching onion plant, or a part thereof, of one or more of the bunching onion varieties disclosed herein, utilizing said plant or plant part as a source of breeding material, and selecting a progeny plant with molecular markers in common with one or more of the bunching onion varieties disclosed herein and/or with morphological and/or physiological characteristics selected from the characteristics of the bunching onion varieties disclosed herein. Breeding steps that may be used include pedigree breeding, backcrossing, mutation breeding, and recurrent selection. As noted above, such breeding methods are known to those of skill in the art. In conjunction with these steps, techniques such as RFLP-enhanced selection, genetic marker enhanced selection (for example, SSR markers), and the making of double haploids may be utilized.

In another aspect the method includes producing a population of progeny bunching onion plants of one or more of the bunching onion varieties disclosed herein, comprising crossing one or more of the onion varieties disclosed herein with another bunching onion plant, thereby producing a population of bunching onion plants, which, on average, derive 50% of their alleles from the bunching onion varieties disclosed herein. A plant of this population may be selected and repeatedly selfed or sibbed with a bunching onion plant resulting from these successive filial generations, or may be backcrossed with a recurrent parent (one or more of the bunching onion varieties disclosed herein). One aspect of this invention is the bunching onion produced by this method and that has obtained at least 50%, at least 60%, at least 70%, at least 80%, at least 90% or more, all subranges and percentages therebetween inclusive, of its alleles from the bunching onion varieties disclosed herein.

One of ordinary skill in the art of plant breeding would know how to evaluate the traits of two plant varieties to determine if there is no significant difference between the two traits expressed by those varieties. Thus, the present invention includes progeny of the bunching onion varieties disclosed herein comprising a combination of at least two traits of the bunching onion varieties disclosed herein, so that said progeny bunching onion plant is not significantly different for said traits than the bunching onion varieties disclosed herein, as determined at the 5% significance level when grown in the same environmental conditions. Using techniques described herein, molecular markers may be used to identify said progeny plant as progeny of one or more of the bunching onion varieties disclosed herein. Mean trait values may be used to determine whether trait differences are significant, and preferably the traits are measured on plants grown under the same environmental conditions.

What is claimed is:

1. A seed of bunching onion variety ‘BGS 390,’ wherein a representative sample of seed of said variety was deposited under NCIMB Accession No. 44000.

2. A bunching onion plant, part thereof, or tissue culture produced by growing the seed of claim 1.

3. A tissue culture produced from protoplasts or cells from the bunching onion plant of claim 2, wherein said cells or protoplasts are produced from a plant part selected from the group consisting of leaf, pollen, embryo, cotyledon, hypocotyl, meristematic cell root, root tip, pistil, anther, ovule, flower, shoot, stem, seed, and petiole.

4. A bunching onion plant regenerated from the tissue culture of claim 3, wherein the regenerated bunching onion plant has all of the morphological and physiological characteristics of variety ‘BGS 390’.

5. A method for producing a bunching onion seed comprising crossing two bunching onion plants and harvesting the resultant bunching onion seed, wherein at least one of the two bunching onion plants is the bunching onion plant of claim 2.

6. The method of claim 5, wherein one of the two bunching onion plants is a bunching onion plant that is unrelated to variety ‘BGS 390’.

7. A bunching onion seed produced by the method of claim 5.

8. A bunching onion plant, part thereof, or tissue culture produced by growing the seed of claim 7.

9. A method of introducing a desired trait into bunching onion variety ‘BGS 390,’ comprising:
(a) crossing a bunching onion plant of variety ‘BGS 390’, wherein a representative sample of seed was deposited under NCIMB Accession No. 44000, with a plant of another bunching onion variety that comprises a desired trait to produce progeny plants, wherein the desired trait is selected from a group consisting of male sterility, herbicide resistance, pest resistance, and resistance to bacterial disease, fungal disease, or viral disease;
(b) selecting one or more progeny plants that have the desired trait to produce selected progeny plants;
(c) crossing the selected progeny plants with the bunching onion plant of variety ‘BGS 390’ to produce backcross progeny plants;
(d) selecting for backcross, progeny plants that have the desired trait and all of the physiological and morphological characteristics of bunching onion variety ‘BGS 390’; and
(e) repeating steps (c) and (d) two or more times in succession to produce selected third or higher backcross progeny plants that comprise the desired trait and all of the physiological and morphological characteristics of bunching onion variety ‘BGS 390’.

10. A bunching onion plant produced by the method of claim 9, wherein said bunching onion plant has the desired trait.

11. The bunching onion plant of claim 10, wherein the desired trait is one or more of herbicide resistance, pest resistance, and male sterility.

12. A seed, plant part, or tissue culture of the bunching onion plant of claim 10.

13. A method of introducing a desired trait into bunching onion variety ‘BGS 390,’ comprising introducing a gene conferring a desired trait into the plant of claim 2.

14. A bunching onion plant produced by the method of claim 13, wherein said bunching onion plant has the desired trait.

15. The bunching onion plant of claim 14, wherein the desired trait is one or more of herbicide resistance, pest resistance, and male sterility.

16. A seed, plant part, or tissue culture of the bunching onion plant of claim 15.
